(19) United States
(12) Patent Application Publication
(10) Pub. No.: US 2025/0019728 A1
(43) Pub. Date: Jan. 16, 2025

(54) METHOD FOR PRODUCING AROMATIC COMPOUND

(71) Applicant: KAO CORPORATION, Tokyo (JP)

(72) Inventors: Jitsuro KANEDA, Wakayama-shi, Wakayama (JP); Fumikazu TAKAHASHI, Wakayama-shi, Wakayama (JP)

(73) Assignee: KAO CORPORATION, Tokyo (JP)

(21) Appl. No.: 18/717,153

(22) PCT Filed: Dec. 7, 2022
(86) PCT No.: PCT/JP2022/045189

§ 371 Date: Jun. 6, 2024

(30) Foreign Application Priority Data
Dec. 7, 2021 (JP) 2021-198834

(51) Int. Cl. C12P 7/42; C12N 9/04; C12N 9/10; C12N 9/88; C12N 15/52

(52) U.S. Cl. CPC C12P 7/42; C12N 9/0006; C12N 9/1022; C12N 9/88; C12N 15/52; C12Y 101/01025; C12Y 202/01001; C12Y 402/01118

(57) ABSTRACT

Provided are a method for producing an aromatic compound or a salt thereof using a transformed cell capable of producing the aromatic compound or the salt thereof, and this transformed cell. The present invention provides a method for producing an aromatic compound or a salt thereof, comprising the step of culturing a transformed cell with enhanced expression of a multi-pass transmembrane polypeptide represented by the following (A) or (B): (A) a polypeptide consisting of the amino acid sequence represented by SEQ ID NO: 2, and (B) a polypeptide consisting of an amino acid sequence having at least 76% identity to the amino acid sequence represented by SEQ ID NO: 2.

### FIELD OF THE INVENTION

The present invention relates to a method for producing an aromatic compound using a transformed cell having the ability to produce the aromatic compound, and the cell.

### BACKGROUND OF THE INVENTION

In recent years, it has been desired to produce useful aromatic compounds including gallic acid using microbes from an inexpensive starting material glucose. In particular, coryneform bacteria (Corynebacterium glutamicum) are useful industrial microbes that have been utilized in the production of various amino acids or nucleic acids. Gene recombination techniques targeting the coryneform bacteria have been established in recent years. It has thus become possible to produce diverse organic compounds including aromatic amino acids such as tyrosine and tryptophan (Non Patent Literature 1), and aromatic compounds such as gallic acid, 4-hydroxybenzoic acid (Non Patent Literature 1), and 4-aminobenzoic acid (Non Patent Literature 2). Among them, gallic acid, because of its strong reducing properties, is used as a photographic developing agent, a starting material for blue ink production, or the like, and its ester such as propyl gallate is used as an antioxidant for fat or oil or butter. Pyrogallol, which is synthesized by the decarboxylation of gallic acid, is used as an electronic material, an organic synthesis reagent, a photographic developing liquid, a mordant for woolen cloth, or the like. Therefore, efficient production of gallic acid is beneficial.

The shikimic acid pathway is an important metabolic pathway through which plants or microbes biosynthesize aromatic compounds. Specifically, phosphoenolpyruvic acid formed in the glycolysis system binds to erythrose 4-phosphate supplied from the pentose phosphate pathway, to thereby provide 3-deoxy-D-arabinoheptulosonic acid 7-phosphate (DAHP), and shikimic acid is produced through 3-dehydroquinic acid (DHQ) and 3-dehydroshikimic acid (DHS). Shikimic acid is further converted to 3-phosphoshikimic acid by the transfer of a phosphate group from adenosine triphosphate, and chorismic acid is produced through 3-phosphoenolpyruvylshikimic acid. In the shikimic acid pathway, a 6-membered carbon ring is formed, followed by the formation of a double bond. Aromatic compounds such as gallic acid, 2,4-pyridinedicarboxylic acid (2,4-PDCA), 2,5-pyridinedicarboxylic acid (2,5-PDCA), catechol, and L-DOPA are produced from protocatechuic acid derived from DHS (FIG. 1).

Meanwhile, multi-pass transmembrane polypeptides belonging to the MFS family (major facilitator superfamily) have been reported to include proteins involved in stress resistance and tolerance (Patent Literature 1). However, it has not been known that these proteins improve the productivity of aromatic compounds.
[Patent Literature 1] U.S. Pat. No. 6,822,084
[Non Patent Literature 1] Metab. Eng. 2018. 50:122-141
[Non Patent Literature 2] Metab. Eng. 2016. 38:322-330

### SUMMARY OF THE INVENTION

The present invention relates to the following.
1) A method for producing an aromatic compound or a salt thereof, comprising the step of culturing a transformed cell with enhanced expression of a multi-pass transmembrane polypeptide represented by the following (A) or (B):
(A) a polypeptide consisting of the amino acid sequence represented by SEQ ID NO: 2, and
(B) a polypeptide consisting of an amino acid sequence having at least 76% identity to the amino acid sequence represented by SEQ ID NO: 2.
2) A transformed cell with enhanced expression of a multi-pass transmembrane polypeptide represented by the following (A) or (B):
(A) a polypeptide consisting of the amino acid sequence represented by SEQ ID NO: 2, and
(B) a polypeptide consisting of an amino acid sequence having at least 76% identity to the amino acid sequence represented by SEQ ID NO: 2, wherein a microbial cell with improved 3-dehydroshikimic acid-producing activity is used as a host.

### BRIEF DESCRIPTION OF DRAWINGS

FIG. 1 is a schematic view illustrating the production pathway of various aromatic compounds in the case of using a coryneform bacterium as a host. In the drawing, aroG and aroF each represent 2-dehydro-3-deoxyarabinoheptonate aldolase; aroB represents 3-dehydroquinate synthase; aroD and qsuC each represent dehydroquinate dehydratase; qsuD represents quinate/shikimate dehydrogenase; aroE3 represents shikimate dehydrogenase; hfm145 represents 3,4-dihydroxybenzoate hydroxylase; qsuB represents dehydroshikimate dehydratase; aroA represents 5-enolpyruvylshikimate-3-phosphate synthase; aroC represents chorismate synthase; and aroK represents shikimate kinase.

FIG. 2 illustrates results of analysis using a transmembrane region prediction program.

### DETAILED DESCRIPTION OF THE INVENTION

The present invention relates to provisions of a method for producing an aromatic compound or a salt thereof using a transformed cell capable of producing the aromatic compound or the salt thereof, and the transformed cell.

The present inventors found that the productivity of aromatic compounds including gallic acid is improved in a transformed cell with enhanced expression of a multi-pass transmembrane polypeptide belonging to the MFS family (major facilitator superfamily), and an aromatic compound or a salt thereof can be efficiently produced using this transformed cell.

The present invention enables an aromatic compound such as protocatechuic acid, gallic acid, 2,4-pyridinedicarboxylic acid, 2,5-pyridinedicarboxylic acid, catechol, L-DOPA, 4-hydroxybenzoic acid, or 4-aminobenzoic acid, or a salt thereof to be efficiently produced by an environmentally friendly fermentation method.

In the present invention, the identity between amino acid sequences or nucleotide sequences is calculated by the Lipman-Pearson method (Science, 1985, 227:1435-1441). Specifically, the identity is calculated by analysis with a unit size to compare (ktup) as 2 using the search homology program of genetic information processing software GENETYX Ver. 12.

In the present invention, the “amino acid sequence obtained by a deletion, substitution, addition, or insertion of one or more amino acids” refers to an amino acid sequence obtained by a deletion, substitution, addition, or insertion of 1 or more and 10 or less, preferably 1 or more and 8 or less, more preferably 1 or more and 5 or less, further more preferably 1 or more and 3 or less amino acids. The “nucleotide sequence obtained by a deletion, substitution, addition, or insertion of one or more nucleotides” refers to a nucleotide sequence obtained by a deletion, substitution, addition, or insertion of 1 or more and 30 or less, preferably 1 or more and 24 or less, more preferably 1 or more and 15 or less, still more preferably 1 or more and 9 or less nucleotides. In the present invention, the “addition” of an amino acid or a nucleotide includes addition of an amino acid or a nucleotide to one end or both ends of a sequence.

In the present invention, the “operable linking” between a control region and a gene means that the gene and the control region are linked such that the gene can be expressed under the control of the control region. The procedures of the “operable linking” between a gene and a control region are well known to those skilled in the art.

In the present invention, the term “original” that is used for a function, a property, or a trait of a cell is used to represent that the function, the property, or the trait is present in a wide type of the cell. By contrast, the term “foreign” is used to represent that the function, the property, or the trait is not pre-existent in the cell and is introduced from the outside. For example, the “foreign” gene or polynucleotide is a gene or a polynucleotide introduced to a cell from the outside. The foreign gene or polynucleotide may be derived from an organism of the same species as that of the recipient cell or may be derived from an organism of different species therefrom (i.e., a heterologous gene or polynucleotide).

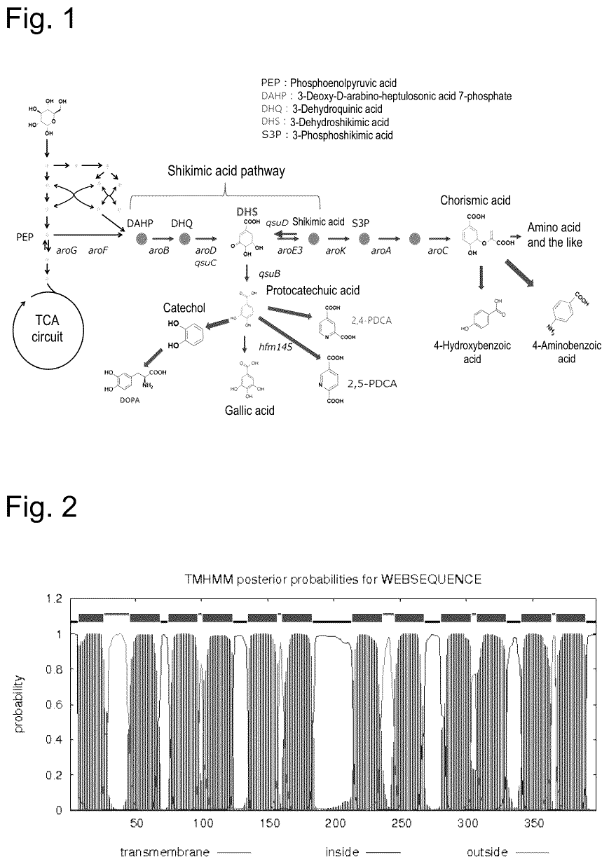

In the present invention, the aromatic compound is an organic aromatic compound that is biosynthesized in a host cell, and specifically refers to an aromatic compound that is synthesized via the shikimic acid pathway, preferably an aromatic compound derived from 3-dehydroshikimic acid (DHS) or chorismic acid (FIG. 1).

Specific examples thereof include protocatechuic acid, catechol, gallic acid, phenylalanine, L-DOPA, tyrosine, pretyrosine, tryptophan, 4-hydroxybenzoic acid, 4-aminobenzoic acid, 2,3-dihydroxybenzoic acid, 2,4-pyridinedicarboxylic acid, 2,5-pyridinedicarboxylic acid, and 4-amino-3-hydroxybenzoic acid.

Among them, the aromatic compound is preferably protocatechuic acid derived from DHS; gallic acid, 2,4-pyridinedicarboxylic acid (2,4-PDCA), 2,5-pyridinedicarboxylic acid (2,5-PDCA), catechol, or L-DOPA derived from protocatechuic acid; 4-hydroxybenzoic acid, 4-aminobenzoic acid, 4-amino-3-hydroxybenzoic acid, tyrosine, or tryptophan derived from chorismic acid; or the like, more preferably protocatechuic acid, an aromatic compound derived from protocatechuic acid (preferably gallic acid or L-DOPA), 4-hydroxybenzoic acid, or 4-amino-3-hydroxybenzoic acid, more preferably gallic acid.

Examples of the salt of the aromatic compound can include base-addition salts and acid-addition salts.

Examples of the base-addition salt include salts with alkali metals such as sodium and potassium, and salts with alkaline earth metals such as calcium and magnesium. Examples of the acid-addition salt include mineral acid salts such as hydrochloride, sulfate, nitrate, and phosphate.

In the present invention, the transformed cell is a cell with enhanced expression of a multi-pass transmembrane polypeptide represented by the following (A) or (B):
(A) a polypeptide consisting of the amino acid sequence represented by SEQ ID NO: 2, and
(B) a polypeptide consisting of an amino acid sequence having at least 76% identity to the amino acid sequence represented by SEQ ID NO: 2.

In this context, the polypeptide consisting of the amino acid sequence represented by SEQ ID NO: 2 in (A) refers to a Corynebacterium glutamicum-derived membrane transport protein belonging to the MFS family (in the present invention, referred to as “GALT0”).

In the polypeptide of (B), the identity to the amino acid sequence represented by SEQ ID NO: 2 is at least 76%, preferably 70% or higher, more preferably 80% or higher, more preferably 85% or higher, more preferably 90% or higher, more preferably 95% or higher, more preferably 96% or higher, more preferably 97% or higher, more preferably 98% or higher, more preferably 99% or higher.

Examples of the amino acid sequence having at least 76% identity to the amino acid sequence represented by SEQ ID NO: 2 include amino acid sequences obtained from the amino acid sequence represented by SEQ ID NO: 2 by a deletion, substitution, addition, or insertion of one or more amino acids.

The polypeptides of (A) and (B) can each be confirmed to have a plurality of transmembrane helix structures by analysis using a transmembrane region prediction program, as shown in Reference Examples mentioned later, and thus presumed to be a “multi-pass transmembrane polypeptide (multiple transmembrane polypeptide)”. In this context, examples of the transmembrane region prediction program include analysis programs using prediction methods such as TMHMM Server, v. 2.0 (Journal of Molecular Biology, 2001, 305:567-580), DAS-TMfilter (Protein Eng., 2002, Volume 15, Issue 9:745-752), and PRED-TMR2 (Protein Eng., 1999, Volume 12, Issue 8:631-634).

As shown in Examples mentioned later, the productivity of aromatic compounds such as gallic acid and protocatechuic acid is improved in cells into which a polynucleotide encoding the polypeptide of (A) or (B) is expressibly introduced. Accordingly, the multi-pass transmembrane polypeptides represented by (A) and (B) are considered to have transporter activity involved in the transport of the aromatic compound or the salt thereof (referred to as “aromatic compound transporter activity”).

Examples of the multi-pass transmembrane polypeptide consisting of an amino acid sequence having at least 76% identity to the amino acid sequence represented by SEQ ID NO: 2 in (B) include the following polypeptides (B1) to (B3):
(B1) a polypeptide consisting of the amino acid sequence represented by SEQ ID NO: 4 or an amino acid sequence having 90% or higher, preferably 92% or higher, more preferably 95% or higher, more preferably 97% or higher, more preferably 98% or higher, more preferably 99% or higher identity to the amino acid sequence,
(B2) a polypeptide consisting of the amino acid sequence represented by SEQ ID NO: 6 or an amino acid sequence having 90% or higher, preferably 92% or higher, more preferably 95% or higher, more preferably 97% or higher, more preferably 98% or higher, more preferably 99% or higher identity to the amino acid sequence, and
(B3) a polypeptide consisting of the amino acid sequence represented by SEQ ID NO: 8 or an amino acid sequence having 90% or higher, preferably 92% or higher, more preferably 95% or higher, more preferably 97% or higher, more preferably 98% or higher, more preferably 99% or higher identity to the amino acid sequence.

In this context, the polypeptide consisting of the amino acid sequence represented by SEQ ID NO: 4 is a Corynebacterium crenatum-derived membrane transport protein and is referred to as “GALT1” in the present invention. The identity between the amino acid sequences of GALT1 and GALT0 is 98%.

The polypeptide consisting of the amino acid sequence represented by SEQ ID NO: 6 is a Corynebacterium glutamicum-derived membrane transport protein and is referred to as “GALT2” in the present invention. The identity between the amino acid sequences of GALT2 and GALT0 is 90%.

The polypeptide consisting of the amino acid sequence represented by SEQ ID NO: 8 is a Corynebacterium crudilactis-derived membrane transport protein and is referred to as “GALT3” in the present invention. The identity between the amino acid sequences of GALT3 and GALT0 is 85.4%.

In addition, a Corynebacterium callunae DSM 20147-derived polypeptide having 75.5% amino acid sequence identity to GALT0 (“GALT4”; amino acid sequence: SEQ ID NO: 10, nucleotide sequence: SEQ ID NO: 9) is known as a membrane transport protein belonging to the MFS family.

Examples of the method for introducing a mutation, such as deletion, substitution, addition, or insertion, of an amino acid to the amino acid sequence of the polypeptide include a method of introducing a mutation, such as deletion, substitution, addition, or insertion, of a nucleotide to a nucleotide sequence encoding the amino acid sequence. Examples of the method of introducing a mutation to the nucleotide sequence include mutation induction with a chemical mutagen such as ethyl methanesulfonate, N-methyl-N-nitrosoguanidine, or nitrous acid or a physical mutagen such as ultraviolet ray, X-ray, gamma ray, or ion beam, site-directed mutagenesis, and methods described by Dieffenbach et al. (Cold Spring Harbar Laboratory Press, New York, 581-621, 1995). Examples of the site-directed mutagenesis include a method using splicing overlap extension (SOE) PCR (Horton et al., Gene 77, 61-68, 1989), the ODA method (Hashimoto-Gotoh et al., Gene, 152, 271-276, 1995), and the Kunkel method (Kunkel, T. A., Proc. Natl. Acad. Sci. USA, 1985, 82, 488). Alternatively, a commercially available kit for site-directed mutagenesis such as Site-Directed Mutagenesis System Mutan-Super Express Km kit (Takara Bio Inc.), Transformer™ Site-Directed Mutagenesis kit (Clonetech Laboratories, Inc.), or KOD-Plus-Mutagenesis Kit (Toyobo Co., Ltd.) may be used.

In the present invention, the transformed cell with enhanced expression of the polypeptide represented by (A) or (B) encompasses cells with an increased expression level of the polypeptide as well as cells with enhanced activity (aromatic compound transporter activity) of the polypeptide. Specifically, the transformed cell is a cell into which a polynucleotide necessary for expressing the polypeptide is expressibly introduced, and the polypeptide may be a foreign one or may be originally carried by the cell. Examples thereof include cells into which the polynucleotide is expressibly introduced, and cells with an enhanced degree of expression of the polynucleotide. Specific examples thereof include cells into which a vector or a DNA fragment containing the polynucleotide and a control region operably linked thereto is introduced, and cells in which the control region of the polynucleotide is substituted by a strong-control region such as a high-expression promoter or an inducible promoter.

In this context, examples of the polynucleotide include polynucleotides encoding the multi-pass transmembrane polypeptide represented by (A) or (B), and preferably include the following polynucleotides (a) and (b) (these polynucleotides are also referred to as the “polynucleotide of the present invention”):
(a) a polynucleotide consisting of the nucleotide sequence represented by SEQ ID NO: 1, and
(b) a polynucleotide consisting of a nucleotide sequence having at least 76% identity to the nucleotide sequence represented by SEQ ID NO: 1.

In this context, the polynucleotide consisting of the nucleotide sequence represented by SEQ ID NO: 1 in (a) refers to a gene encoding the multi-pass transmembrane polypeptide GALT0 mentioned above (cg3038).

In the polynucleotide of (b), the identity to the nucleotide sequence represented by SEQ ID NO: 1 is at least 76%, preferably 80% or higher, more preferably 85% or higher, more preferably 90% or higher, more preferably 95% or higher, more preferably 96% or higher, more preferably 978 or higher, more preferably 98% or higher, more preferably 99% or higher.

Examples of the nucleotide sequence having at least 76% identity to the nucleotide sequence represented by SEQ ID NO: 1 include nucleotide sequences obtained from the nucleotide sequence represented by SEQ ID NO: 1 by a deletion, substitution, addition, or insertion of one or more nucleotides. The method for introducing a mutation, such as deletion, substitution, addition, or insertion, of a nucleotide to the nucleotide sequence is as mentioned above. The polynucleotide can be in a single-stranded or double-stranded form and may be DNA or RNA. The DNA can be cDNA or artificial DNA such as chemically synthetic DNA.

Examples of the polynucleotide consisting of a nucleotide sequence having at least 76% identity to the nucleotide sequence represented by SEQ ID NO: 1 in (b) include the following polynucleotides (b1) to (b3):
(b1) a polynucleotide consisting of the nucleotide sequence represented by SEQ ID NO: 3 or a nucleotide sequence having 90% or higher, preferably 92% or higher, more preferably 95% or higher, more preferably 97% or higher, more preferably 98% or higher, more preferably 99% or higher identity to the nucleotide sequence,
(b2) a polynucleotide consisting of the nucleotide sequence represented by SEQ ID NO: 5 or a nucleotide sequence having 90% or higher, preferably 92% or higher, more preferably 95% or higher, more preferably 97% or higher, more preferably 98% or higher, more preferably 99% or higher identity to the nucleotide sequence, and
(b3) a polynucleotide consisting of the nucleotide sequence represented by SEQ ID NO: 7 or a nucleotide sequence having 90% or higher, preferably 92% or higher, more preferably 95% or higher, more preferably 97% or higher, more preferably 98% or higher, more preferably 99% or higher identity to the nucleotide sequence.

In this context, the polynucleotide consisting of the nucleotide sequence represented by SEQ ID NO: 3 is a polynucleotide encoding the GALT1, the polynucleotide consisting of the nucleotide sequence represented by SEQ ID NO: 5 is a polynucleotide encoding the GALT2, and the polynucleotide consisting of the nucleotide sequence represented by SEQ ID NO: 7 is a polynucleotide encoding the GALT3.

The polynucleotide may be incorporated in a vector. Preferably, the vector containing the polynucleotide of the present invention is an expression vector. Preferably, the vector is an expression vector that can introduce the polynucleotide of the present invention into a host microbe and enables the polynucleotide to be expressed in the host microbe. Preferably, the vector contains the polynucleotide of the present invention and a control region operably linked thereto. The vector may be a vector capable of extrachromosomally proliferating and replicating autonomously, or may be a vector that is integrated into a chromosome.

Specific examples of the vector include pBluescript II SK(−) (Stratagene), pUC series of vectors such as pUC18/19 and pUC118/119 (Takara Bio Inc.), pET series of vectors (Takara Bio Inc.), pGEX series of vectors (GE Healthcare), pCold series of vectors (Takara Bio Inc.), pHY300PLK (Takara Bio Inc.), pUB110 (Mckenzie, T. et al., 1986, Plasmid 15 (2): 93-103), pBR322 (Takara Bio Inc.), pRS403 (Stratagene), pMW218/219 (Nippon Gene Co., Ltd.), pRI series of vectors such as pRI909/910 (Takara Bio Inc.), pBI series of vectors (Clontech Laboratories, Inc.), IN3 series of vectors (Inplanta Innovations Inc.), pPTR1/2 (Takara Bio Inc.), pDJB2 (D. J. Ballance et al., Gene, 36, 321-331, 1985), pAB4-1 (van Hartingsveldt W et al., Mol Gen Genet, 206, 71-75, 1987), pLeu4 (M. I. G. Roncero et al., Gene, 84, 335-343, 1989), pPyr225 (C. D. Skory et al., Mol Genet Genomics, 268, 397-406, 2002), and pFG1 (Gruber, F. et al., Curr Genet, 18, 447-451, 1990).

The polynucleotide may be constructed as a DNA fragment containing this polynucleotide. Examples of the DNA fragment include DNA fragments amplified by PCR and DNA fragments cleaved with restriction enzymes. Preferably, the DNA fragment can be an expression cassette containing the polynucleotide of the present invention and a control region operably linked thereto.

The control region contained in the vector or the DNA fragment is a sequence for expressing the polynucleotide of the present invention in a host cell into which the vector or the DNA fragment is introduced. Examples thereof include expression regulation regions such as promoters and terminators, and replication origins. The type of the control region can be appropriately selected depending on the type of the host microbe into which the vector or the DNA fragment is introduced. If necessary, the vector or the DNA fragment may further have a selective marker such as an antibiotic resistance gene or an amino acid synthesis-related gene.

A general transformation method, for example, an electroporation method, a transformation method, a transfection method, a conjugation method, a protoplast method, a particle gun method, or an Agrobacterium method, can be used in the introduction of the vector or the DNA fragment into the host cell.

The transformed cell into which the vector or the DNA fragment of interest is introduced can be selected through the use of a selective marker. When the selective marker is, for example, an antibiotic resistance gene, the cell into which the vector or the DNA fragment of interest is introduced can be selected by culture in a medium supplemented with the antibiotic. When the selective marker is, for example, an amino acid synthesis-related gene, the cell into which the vector or the DNA fragment of interest is introduced can be selected by introducing a gene into a host cell that requires the amino acid, and then using the presence or absence of the amino acid auxotrophy as an index. Alternatively, the introduction of the vector or the DNA fragment of interest can also be confirmed by examining the DNA sequence of the transformed cell by PCR or the like.

Examples of the strong-control region include, but are not particularly limited to, high-expression promoters known in the art such as T7 promoter, lac promoter, tac promoter, trp promoter, tu promoter, and gap promoter.

Alternatively, a prokaryote-derived inducible promoter can be used as the strong-control region. Examples thereof include, but are not particularly limited to, vanA (cg2616) promoter which is induced by the addition of ferulic acid, vanillic acid or vanillin, rhcH promoter which is induced by the addition of resorcinol or 2,4-dihydroxybenzoic acid, pcaI promoter which is induced by the addition of 4-hydroxybenzoic acid, nagI (cg3351) gene promoter which is induced by the addition of 3-hydroxybenzoic acid, benA (cg2637) gene promoter (hereinafter, abbreviated to Pben) which is induced by the addition of benzoic acid, and cg2118 gene promoter and ptsS (cg2925) gene promoter which are induced by the addition of either fructose or sucrose.

Examples of the method for substituting the control region of the polynucleotide residing on the genome of the host cell by the strong-control region include methods of introducing a DNA fragment containing polynucleotide sequences of the strong-control region and a selective marker into the host cell, and selecting a cell transformed by homologous recombination or nonhomologous recombination.

In the present invention, the host cell may not be limited as long as the cell is suitable for the production of the aromatic compound or a salt thereof. Any of microbial cells, plant cells, and animal cells may be used. A microbial cell is preferred.

A microbial cell with improved 3-dehydroshikimic acid-producing activity is further more preferably used as the host cell from the viewpoint of the production efficiency of the aromatic compound or a salt thereof, particularly, from the viewpoint of the production efficiency of an aromatic compound derived from 3-dehydroshikimic acid, or a salt thereof, such as protocatechuic acid, gallic acid, shikimic acid, 2,4-pyridinedicarboxylic acid, 2,5-pyridinedicarboxylic acid, catechol, L-DOPA, chorismic acid, 4-hydroxybenzoic acid, 4-aminobenzoic acid, or 4-amino-3-hydroxybenzoic acid.

The microbial cell that can be used may be from any of E. coli, Bacillus subtilis, actinomycetes, bacteria of the genus Pseudomonas, bacteria of the genus Streptococcus, bacteria of the genus Lactobacillus, fungi (the genus Neurospora, the genus Aspergillus, the genus Trichoderma, and the like), yeasts (the genus Saccharomyces, the genus Kluyveromyces, the genus Schizosaccharomyces, the genus Yarrowia, the genus Trichosporon, the genus Rhodosporidium, the genus Pichia, the genus Candida, and the like), and the like, and is preferably a prokaryotic microbial cell, more preferably from a gram-positive bacterium, further more preferably from an actinomycete.

The actinomycete is preferably a group of microbes defined as coryneform bacteria (Bergey's Manual of Determinative Bacteriology, Vol. 8, 599 (1974)). Specific examples thereof include bacteria of the genus Corynebacterium, bacteria of the genus Brevibacterium, bacteria of the genus Arthrobacter, bacteria of the genus Mycobacterium, bacteria of the genus Rhodococcus, bacteria of the genus Streptomyces, and bacteria of the genus Micrococcus.

Examples of the bacterium of the genus Corynebacterium include Corynebacterium glutamicum, Corynebacterium efficiens, Corynebacterium ammoniagenes, Corynebacterium halotolerance, Corynebacterium alkanolyticum, Corynebacterium crenatum, Corynebacterium crudilactis, and Corynebacterium callunae.

Examples of the bacterium of the genus Brevibacterium include Brevibacterium ammoniagenes.

Examples of the bacterium of the genus Arthrobacter include Arthrobacter globiformis.

Examples of the bacterium of the genus Mycobacterium include Mycobacterium bovis. Examples of the bacterium of the genus Micrococcus include Micrococcus freudenreichii, Micrococcus leuteus, Micrococcus ureae, and Micrococcus roseus.

Among the coryneform bacteria, a bacterium of the genus Corynebacterium is preferred, and Corynebacterium glutamicum is more preferred.

The microbial cell may be a wild-type strain or may be a mutant or an artificial genetic recombinant thereof.

Examples of the microbial cell with improved 3-dehydroshikimic acid-producing activity include microbial cells with an enhanced gene necessary for producing 3-dehydroshikimic acid, and specifically include microbial cells subjected to any one or more of the genetic manipulations (i), (ii), (iii), and (iv) given below, preferably microbial cells subjected to any two or more of the genetic manipulations (i), (ii), (iii), and (iv), more preferably microbial cells subjected to any three or more of the genetic manipulations (i), (ii), (iii), and (iv), further more preferably microbial cells subjected to all the genetic manipulations (i), (ii), (iii), and (iv).

In this context, the enhancement of a gene encompasses the introduction of a predetermined gene in an expressible state, and the introduction of a mutation to a predetermined gene or a control region of the gene.
(i) Enhancement of one or more genes selected from the group consisting of dehydroshikimate dehydratase gene, dehydroquinate dehydratase gene, quinate dehydrogenase gene, and shikimate dehydrogenase gene.
(ii) Enhancement of one or more genes selected from a gene group involved in the shikimic acid synthesis pathway consisting of 2-dehydro-3-deoxyarabinoheptonate aldolase gene, 3-dehydroquinate synthase gene, and shikimate dehydrogenase gene.
(iii) Enhancement of one or more genes selected from a gene group involved in the pentose phosphate pathway consisting of glucose-6-phosphate dehydrogenase gene, 6-phosphogluconolactonase gene, phosphogluconate dehydrogenase gene, ribose-5-phosphate isomerase gene, ribulose-5-phosphate-3-epimerase gene, transketolase gene, and transaldolase gene.
(iv) Enhancement of a gene encoding a polypeptide having 3,4-dihydroxybenzoate hydroxylase activity.

In the prepared transformant, the enhanced expression of the multi-pass transmembrane polypeptide represented by (A) or (B) can be confirmed from, for example, an improved transcript level of a gene encoding the polypeptide in the transformant compared with its host cell (parent cell). The transcript level of the gene can be measured by mRNA level measurement by quantitative PCR, RNA-Seq analysis using a next-generation sequencer, DNA microarray analysis, or the like.

A cell producing a useful aromatic compound or salt thereof can be obtained by culturing the prepared transformed cell, evaluating the productivity of the aromatic compound or a salt thereof, and selecting a proper transformed cell. The method for measuring a product can be performed in accordance with a method described in Reference Examples mentioned later.

The method for producing the aromatic compound or a salt thereof according to the present invention is carried out by culturing the transformed cell mentioned above, preferably in the presence of a saccharide, and collecting the aromatic compound or the salt thereof of interest.

The saccharide is preferably glucose. A monosaccharide such as fructose, mannose, arabinose, xylose, or galactose as well as a saccharide capable of producing glucose by metabolism can also be used. Such a saccharide includes an oligosaccharide or a polysaccharide having a glucose unit. Examples thereof include: disaccharides such as cellobiose, sucrose, lactose, maltose, trehalose, cellobiose, and xylobiose; and polysaccharides such as dextrin and soluble starches.

For example, molasses can also be used as a starting material containing these starting compounds. Alternatively, a saccharified solution containing a plurality of saccharides such as glucose can also be used which is obtained by saccharifying a nonedible agricultural waste such as straw (rice straw, barley straw, wheat straw, rye straw, oat straw, or the like), bagasse, or corn stover, an energy crop such as switchgrass, napier grass, or Miscanthus, wood chips, used paper, or the like with a diastatic enzyme or the like.

Any of natural media and synthetic media may be used as a medium for culturing the transformed cell as long as the medium contains a carbon source, a nitrogen source, an inorganic salt, and the like and permits efficient culture of the transformed cell of the present invention.

The saccharide described above or molasses or a saccharified solution containing the saccharide is used as the carbon source. Examples of the carbon source that can be used, in addition to the saccharide described above, include: sugar alcohols such as mannitol, sorbitol, xylitol, and glycerin; organic acids such as acetic acid, citric acid, lactic acid, fumaric acid, maleic acid, and gluconic acid; alcohols such as ethanol and propanol; and hydrocarbons such as normal paraffin. These carbon atoms can be used singly or as a mixture of two or more thereof.

The concentration of the starting compound saccharide in the culture solution is preferably from 1 to 20 w/v %, more preferably from 2 to 10 w/v %, further more preferably from 2 to 5 w/v %.

Examples of the nitrogen source that can be used include peptone, meat extracts, yeast extracts, casein hydrolysates, alkali extracts of soymeal, alkylamines such as methylamine, nitrogen-containing organic compounds such as amino acids, ammonia and salts thereof (inorganic or organic ammonium compounds such as ammonium chloride, ammonium sulfate, ammonium nitrate, and ammonium acetate), urea, ammonia water, sodium nitrate, and potassium nitrate.

Examples of the inorganic salt include primary potassium phosphate, secondary potassium phosphate, magnesium sulfate, sodium chloride, ferrous sulfate, manganese sulfate, zinc sulfate, cobalt sulfate, and calcium carbonate.

The medium may be further supplemented, if necessary, with a vitamin, an antifoaming agent, and the like. Examples of the vitamin include biotin, thiamine (vitamin B1), pyridoxin (vitamin B6), pantothenic acid, inositol, and nicotinic acid.

Examples of the medium for coryneform bacteria include A medium [J. Mol. Microbiol. Biotechnol. 7:182-196 (2004)], BT medium [J. Mol. Microbiol. Biotechnol. 8:91-103 (2004)], and CGXII medium [JP-B-6322576]. These media can be used with a saccharide concentration set to the range described above.

Prior to the reaction or culture involving the saccharide, the transformant is preferably allowed to proliferate by culture at a temperature of from approximately 25 to 38° C. for approximately 12 to 48 hours under aerobic conditions in the same medium as above.

The culture temperature or the reaction temperature is preferably from 15 to 45° C., more preferably from 25 to 37° C.

The culture or reaction time is from 24 hours to 168 hours, preferably from 24 hours to 96 hours, more preferably from 24 hours to 72 hours. The culture or the reaction can be performed, if necessary, with stirring or shaking. During the culture, an antibiotic such as ampicillin or kanamycin may be added, if necessary, to the medium.

The culture may be any of batch, feeding, and continuous processes and is preferably a batch process.

The culture or the reaction may be performed under aerobic conditions or may be performed under reductive conditions and is preferably performed under aerobic conditions.

In the case of performing the reaction or the culture under aerobic conditions, the reaction or the culture is preferably performed under conditions that suppress excessive proliferation of the transformant, from the viewpoint of the production efficiency of the aromatic compound or a salt thereof.

If the aromatic compound is susceptible to oxidation, the culture is preferably performed under conditions having a low dissolved oxygen concentration. For example, in the production of gallic acid, specifically, the dissolved oxygen concentration is preferably from 0.1 to 3 ppm, more preferably from 0.1 to 1 ppm.

A method for collecting and purifying the aromatic compound or a salt thereof from the cultures is not particularly limited. Specifically, the method can be carried out by a combination of a well-known ion-exchange resin method, a precipitation method, a crystallization method, a recrystallization method, a concentration method, and other methods. The aromatic compound or a salt thereof can be obtained, for example, by removing bacterial cells by centrifugation or the like, and then removing ionic substances through cation- and anion-exchange resins, followed by concentration. The aromatic compound or a salt thereof accumulated in the cultures may be used directly without being isolated.

In relation to the embodiments mentioned above, the present invention further discloses the following aspects:
<1> A method for producing an aromatic compound or a salt thereof, comprising the step of culturing a transformed cell with enhanced expression of a multi-pass transmembrane polypeptide represented by the following (A) or (B):
(A) a polypeptide consisting of the amino acid sequence represented by SEQ ID NO: 2, and
(B) a polypeptide consisting of an amino acid sequence having at least 76% identity to the amino acid sequence represented by SEQ ID NO: 2.
<2> The method according to <1>, wherein the polypeptide consisting of an amino acid sequence having at least 76% identity to the amino acid sequence represented by SEQ ID NO: 2 in (B) is a polypeptide represented by any of the following (B1) to (B3):
(B1) a polypeptide consisting of the amino acid sequence represented by SEQ ID NO: 4 or an amino acid sequence having 90% or higher identity to the amino acid sequence,
(B2) a polypeptide consisting of the amino acid sequence represented by SEQ ID NO: 6 or an amino acid sequence having 90% or higher identity to the amino acid sequence, and
(B3) a polypeptide consisting of the amino acid sequence represented by SEQ ID NO: 8 or an amino acid sequence having 90% or higher identity to the amino acid sequence.
<3> The method according to <1> or <2>, wherein a polynucleotide encoding the multi-pass transmembrane polypeptide represented by (A) or (B) is contained in an expressible state.
<4> The method according to <3>, wherein the polynucleotide encoding the multi-pass transmembrane polypeptide represented by (A) or (B) is a polynucleotide represented by the following (a) or (b):
(a) a polynucleotide consisting of the nucleotide sequence represented by SEQ ID NO: 1, and
(b) a polynucleotide consisting of a nucleotide sequence having at least 76% identity to the nucleotide sequence represented by SEQ ID NO: 1.
<5> The method according to <4>, wherein the polynucleotide consisting of a nucleotide sequence having at least 76% identity to the nucleotide sequence represented by SEQ ID NO: 1 in (b) is a polynucleotide represented by any of the following (b1) to (b3):
(b1) a polynucleotide consisting of the nucleotide sequence represented by SEQ ID NO: 3 or a nucleotide sequence having 90% or higher identity to the nucleotide sequence,
(b2) a polynucleotide consisting of the nucleotide sequence represented by SEQ ID NO: 5 or a nucleotide sequence having 90% or higher identity to the nucleotide sequence, and
(b3) a polynucleotide consisting of the nucleotide sequence represented by SEQ ID NO: 7 or a nucleotide sequence having 90% or higher identity to the nucleotide sequence.
<6> The method according to any of <1> to <5>, wherein the culture is performed in the presence of a saccharide.
<7> The method according to any of <1> to <6>, wherein a host of the transformed cell is a microbial cell with improved 3-dehydroshikimic acid-producing activity.
<8> The method according to <7>, wherein the microbial cell with improved 3-dehydroshikimic acid-producing activity is a cell subjected to any one or more of the following genetic manipulations (i), (ii), (iii), and (iv):
(i) enhancement of one or more genes selected from the group consisting of dehydroshikimate dehydratase gene, dehydroquinate dehydratase gene, quinate dehydrogenase gene, and shikimate dehydrogenase gene,
(ii) enhancement of one or more genes selected from a gene group involved in the shikimic acid synthesis pathway consisting of 2-dehydro-3-deoxyarabinoheptonate aldolase gene, 3-dehydroquinate synthase gene, and shikimate dehydrogenase gene,
(iii) enhancement of one or more genes selected from a gene group involved in the pentose phosphate pathway consisting of glucose-6-phosphate dehydrogenase gene, 6-phosphogluconolactonase gene, phosphogluconate dehydrogenase gene, ribose-5-phosphate isomerase gene, ribulose-5-phosphate-3-epimerase gene, transketolase gene, and transaldolase gene, and
(iv) enhancement of a gene encoding a polypeptide having 3,4-dihydroxybenzoate hydroxylase activity.
<9> The method according to <8>, wherein the microbial cell with improved 3-dehydroshikimic acid-producing activity is a microbial cell subjected to any two or more of the genetic manipulations (i), (ii), (iii), and (iv).
<10> The method according to <8>, wherein the microbial cell with improved 3-dehydroshikimic acid-producing activity is a microbial cell subjected to any three or more of the genetic manipulations (i), (ii), (iii), and (iv).
<11> The method according to <8>, wherein the microbial cell with improved 3-dehydroshikimic acid-producing activity is a microbial cell subjected to the genetic manipulations (i), (ii), (iii), and (iv).
<12> The method according to any of <7> to <11>, wherein the microbial cell is a coryneform bacterium.
<13> The method according to <12>, wherein the coryneform bacterium is a bacterium of the genus Corynebacterium.
<14> The method according to <13>, wherein the bacterium of the genus Corynebacterium is Corynebacterium glutamicum, Corynebacterium efficiens, Corynebacterium ammoniagenes, Corynebacterium halotolerance, Corynebacterium alkanolyticum, Corynebacterium crenatum, Corynebacterium crudilactis, or Corynebacterium callunae.
<15> The method according to <13>, wherein the bacterium of the genus Corynebacterium is Corynebacterium glutamicum.
<16> The method according to any of <1> to <15>, wherein the aromatic compound or the salt thereof is an aromatic compound derived from 3-dehydroshikimic acid or a salt thereof.
<17> The method according to <16>, wherein the aromatic compound or the salt thereof is gallic acid, protocatechuic acid, catechol, L-DOPA, 2,4-pyridinedicarboxylic acid, 2,5-pyridinedicarboxylic acid, 4-hydroxybenzoic acid, 4-aminobenzoic acid, 4-amino-3-hydroxybenzoic acid, or a salt thereof.
<18> The method according to <15>, wherein the aromatic compound or the salt thereof is gallic acid, protocatechuic acid, L-DOPA, 4-hydroxybenzoic acid, 4-amino-3-hydroxybenzoic acid, or a salt thereof.
<19> The method according to <18>, wherein the aromatic compound or the salt thereof is gallic acid, protocatechuic acid, or a salt thereof.
<20> A transformed cell with enhanced expression of a multi-pass transmembrane polypeptide represented by the following (A) or (B):
(A) a polypeptide consisting of the amino acid sequence represented by SEQ ID NO: 2, and
(B) a polypeptide consisting of an amino acid sequence having at least 76% identity to the amino acid sequence represented by SEQ ID NO: 2, wherein a microbial cell with improved 3-dehydroshikimic acid-producing activity is used as a host.
<21> The transformed cell according to <20>, wherein the microbial cell with improved 3-dehydroshikimic acid-producing activity is a cell subjected to any one or more of the following genetic manipulations (i), (ii), (iii), and (iv):
(i) enhancement of one or more genes selected from the group consisting of dehydroshikimate dehydratase gene, dehydroquinate dehydratase gene, quinate dehydrogenase gene, and shikimate dehydrogenase gene,
(ii) enhancement of one or more genes selected from a gene group involved in the shikimic acid synthesis pathway consisting of 2-dehydro-3-deoxyarabinoheptonate aldolase gene, 3-dehydroquinate synthase gene, and shikimate dehydrogenase gene,
(iii) enhancement of one or more genes selected from a gene group involved in the pentose phosphate pathway consisting of glucose-6-phosphate dehydrogenase gene, 6-phosphogluconolactonase gene, phosphogluconate dehydrogenase gene, ribose-5-phosphate isomerase gene, ribulose-5-phosphate-3-epimerase gene, transketolase gene, and transaldolase gene, and
(iv) enhancement of a gene encoding a polypeptide having 3,4-dihydroxybenzoate hydroxylase activity.
<22> The transformed cell according to <21>, wherein the microbial cell with improved 3-dehydroshikimic acid-producing activity is a microbial cell subjected to any two or more of the genetic manipulations (i), (ii), (iii), and (iv).
<23> The transformed cell according to <21>, wherein the microbial cell with improved 3-dehydroshikimic acid-producing activity is a microbial cell subjected to any three or more of the genetic manipulations (i), (ii), (iii), and (iv).
<24> The transformed cell according to <21>, wherein the microbial cell with improved 3-dehydroshikimic acid-producing activity is a microbial cell subjected to the genetic manipulations (i), (ii), (iii), and (iv).
<25> The transformed cell according to any of <20> to <24>, wherein the polypeptide consisting of an amino acid sequence having at least 76% identity to the amino acid sequence represented by SEQ ID NO: 2 in (B) is a polypeptide represented by any of the following (B1) to (B3):
(B1) a polypeptide consisting of the amino acid sequence represented by SEQ ID NO: 4 or an amino acid sequence having 90% or higher identity to the amino acid sequence,
(B2) a polypeptide consisting of the amino acid sequence represented by SEQ ID NO: 6 or an amino acid sequence having 90% or higher identity to the amino acid sequence, and
(B3) a polypeptide consisting of the amino acid sequence represented by SEQ ID NO: 8 or an amino acid sequence having 90% or higher identity to the amino acid sequence.
<26> The transformed cell according to according to any of <20> to <25>, wherein a polynucleotide encoding the multi-pass transmembrane polypeptide represented by (A) or (B) is contained in an expressible state.
<27> The transformed cell according to <26>, wherein the polynucleotide encoding the multi-pass transmembrane polypeptide represented by (A) or (B) is a polynucleotide represented by the following (a) or (b):
(a) a polynucleotide consisting of the nucleotide sequence represented by SEQ ID NO: 1, and
(b) a polynucleotide consisting of a nucleotide sequence having at least 76% identity to the nucleotide sequence represented by SEQ ID NO: 1.
<28> The transformed cell according to <27>, wherein the polynucleotide consisting of a nucleotide sequence having at least 76% identity to the nucleotide sequence represented by SEQ ID NO: 1 in (b) is a polynucleotide represented by any of the following (b1) to (b3):
(b1) a polynucleotide consisting of the nucleotide sequence represented by SEQ ID NO: 3 or a nucleotide sequence having 90% or higher identity to the nucleotide sequence,
(b2) a polynucleotide consisting of the nucleotide sequence represented by SEQ ID NO: 5 or a nucleotide sequence having 90% or higher identity to the nucleotide sequence, and
(b3) a polynucleotide consisting of the nucleotide sequence represented by SEQ ID NO: 7 or a nucleotide sequence having 90% or higher identity to the nucleotide sequence.
<29> The transformed cell according to any of <20> to <28>, wherein the microbial cell is a coryneform bacterium.
<30> The transformed cell according to <29>, wherein the coryneform bacterium is a bacterium of the genus Corynebacterium.
<31> The transformed cell according to <30>, wherein the bacterium of the genus Corynebacterium is Corynebacterium glutamicum, Corynebacterium efficiens, Corynebacterium ammoniagenes, Corynebacterium halotolerance, Corynebacterium alkanolyticum, Corynebacterium crenatum, Corynebacterium crudilactis, or Corynebacterium callunae.
<32> The transformed cell according to <31>, wherein the bacterium of the genus Corynebacterium is Corynebacterium glutamicum.

### Examples

Hereinafter, the present invention will be described in more detail with reference to Examples. However, the technical scope of the present invention is not limited by the following Examples.

### (1) Preparation of Gallic Acid (Also Referred to as GAL)-Producing Bacterium

### 1) Construction of Plasmid for Substituting Cg0620 Gene Region by Gene of Polypeptide Having 3,4-Dihydroxybenzoate Hydroxylase Activity

Nucleotide positions described in Examples given below are nucleotide positions in the genomic sequence of an ATCC13032 strain. Information on the genomic sequence was obtained from the GB database of NCBI under accession No. NC_006958.

The enzyme for PCR used was PrimeSTAR Max DNA Polymerase (Takara Bio Inc.).

The genomic DNA of the ATCC13032 strain (=NBRC 12168 strain) was used as a template in amplification using primers OT20 and OT21 to obtain a 5′ DNA fragment of the cg0620 gene region. Also, the genomic DNA was used as a template in amplification using primers OT23 and OT24 to obtain a 3′ DNA fragment of the cg0620 gene region. A DNA fragment (OT25) containing tuf gene (cg0587) promoter (hereinafter, referred to as tu promoter) carried by the Corynebacterium glutamicum ATCC13032 strain was prepared by artificial gene synthesis. This DNA fragment was used as a template in amplification using primers OT26 and OT27 to obtain a DNA fragment of the promoter region. Two types of DNA fragments (SEQ ID NOS: 11 and 12) containing a gene (hereinafter, abbreviated to hfm145VF) of a polypeptide having 3,4-dihydroxybenzoate hydroxylase activity were prepared by artificial gene synthesis. These DNA fragments were separately used as templates in amplification using two types of DNA primers (OT30 and OT31; and OT32 and OT33), respectively, to obtain two types of DNA fragments. A vector fragment was amplified with pHKPsacB1 as a template using primers OT34 and OT35. The obtained PCR products were treated with DpnI (Takara Bio Inc.). The respective DNA fragments were purified from the obtained six types of PCR products using NucleoSpin Gel and PCR Clean-up (Takara Bio Inc.) and ligated using In-Fusion HD Cloning Kit (Takara Bio Inc.) to obtain a plasmid pHKPsacB_cg0620-Ptu-hfm145VF-hfm145VFopt. The ECOS Competent E. Coli DH5a strain (Nippon Gene Co., Ltd.) was transformed with the obtained plasmid solution, and the cell solution was spread over LB agar medium containing kanamycin and left standing overnight at 37° C. Transformants having the plasmid were inoculated to 2 mL of LB liquid medium containing kanamycin and cultured overnight at 37° C. The plasmid was purified from this culture solution using NucleoSpin Plasmid EasyPure (Takara Bio Inc.) to obtain pHKPsacB_cg0620-Ptu-hfm145VF-hfm145VFopt.

### 2) Preparation of Strain Harboring Gene of Polypeptide Having 3,4-Dihydroxybenzoate Hydroxylase Activity

The plasmid pHKPsacB_cg0620-Ptu-hfm145VF-hfm145VFopt mentioned above was introduced to a CY44 strain (the CY44 strain is the tkt strain described in Reference Example 14 of JP-B-6322576 in which the expression of transketolase (also referred to as tkt) gene is enhanced by transcriptional control through tu promoter; the transcription of dehydroshikimate dehydratase (also referred to as qsuB) gene and vanR (cg2615) gene can be induced by the addition of benzoic acid; and shikimate dehydrogenase (also referred to as aroE3) gene is controlled by VanR repressor) by use of the transformation method by electroporation (Bio-Rad Laboratories, Inc.). A KC148sr strain was obtained by selection based on kanamycin resistance. As a result of analyzing the KC148sr strain by PCR (Sapphire Amp (Takara Bio Inc.)) using primers OT20 and OT36, predictable results were obtained. The KC148sr strain was therefore confirmed to be a single-crossover homologous recombinant in which the plasmid pHKPsacB_cg0620-Ptu-hfm145VF-hfm145VFopt was introduced into the cg0620 gene region.

The KC148sr strain was cultured in 1 mL of LB liquid medium (10 g/L tryptone, 5 g/L yeast extracts, and 10 g/L sodium chloride) for 24 hours, and a portion of the culture solution was smear-cultured on LB agar medium containing 20% sucrose to obtain a KC148 strain. The KC148 strain was confirmed to be a double-crossover homologous recombinant in the Ptu-hfm145VF-hfm145VFop gene was introduced into the cg0620 gene region, as expected, by PCR (Sapphire Amp (Takara Bio Inc.)) using primers OT36 and OT37.

### (2) Preparation of Lactate Dehydrogenase Gene (Hereinafter, Also Referred to as Ldh Gene) Disruptant

### 1) Preparation of Plasmid for Disrupting Ldh (Cg3219) Gene

The enzyme for PCR used was PrimeSTAR Max DNA Polymerase (Takara Bio Inc.). A vector fragment was amplified with pHKPsacB1 (described in JP-B-6322576) as a template using primers pHKPsacB-F2 and pHKPsacB-R2. The genomic DNA of the ATCC13032 strain (=NBRC 12168 strain) was used as a template in amplification using primers 3219-up-F and 3219-up-R to obtain a 5′ DNA fragment of the cg3219 gene, and the genomic DNA was used as a template in amplification using primers 3219-down-F and 3219-down-R to obtain a 3′ DNA fragment of the cg3219 gene. The obtained PCR products were treated with DpnI (Takara Bio Inc.). The respective DNA fragments were purified from the obtained three types of PCR products using NucleoSpin Gel and PCR Clean-up (Takara Bio Inc.) and then ligated using In-Fusion HD Cloning Kit (Clontech Laboratories, Inc.) to prepare pHKBsacB-Δldh. The ECOS Competent E. Coli DH5a strain (Nippon Gene Co., Ltd.) was transformed with the obtained plasmid solution, and the cell solution was spread over LB agar medium containing kanamycin and left standing overnight at 37° C. The obtained colony was used as a template in colony PCR using Sapphire Amp (Takara Bio Inc.) as an enzyme. The primers used were 3219-up-F and 3219-down-R, and the introduction of the DNA fragment of interest was confirmed. Transformants having the plasmid confirmed to harbor the gene were inoculated to 2 mL of LB liquid medium containing kanamycin and cultured overnight at 37° C. The plasmid was purified from this culture solution using NucleoSpin Plasmid EasyPure (Takara Bio Inc.) to obtain pHKBsacB-Δldh.

### 2) Obtainment of Ldh Gene (Cg3219) Disruptant

KC148 was transformed with the plasmid pHKBsacB-Δldh obtained as described above by electroporation (Bio-Rad Laboratories, Inc.). KC148Δldh-sr was obtained by selection based on kanamycin resistance. As a result of performing PCR (Sapphire Amp) with the obtained colony as a template using primers sacB-1 and 3219-up1500, predictable results were obtained. The plasmid pHKBsacB-Δldh was therefore confirmed to be introduced into the cg3219 gene region by single-crossover homologous recombination. KC148Δldh-sr was cultured in 1 mL of LB liquid medium for 24 hours, and a portion of the culture solution was smear-cultured on LB agar medium containing 20% sucrose to obtain a KC148Δldh strain. The ldh gene (cg3219) was confirmed to be deleted by double-crossover homologous recombination, by colony PCR (Sapphire Amp) using primers 3219-coloP-F and 3219-coloP-R. In addition, the kanamycin resistance gene and the sacB gene were confirmed to be also deleted.

(3) Preparation of Strain with GALT0 Gene Knocked in Cg3219 Locus

### 1) Preparation of Plasmid for Knocking GALT0 Gene in Cg3219 Locus

The enzyme for PCR used was PrimeSTAR Max DNA Polymerase (Takara Bio Inc.). A vector fragment was amplified with pHKBsacB-Δldh as a template using primers ocJK83 and ocJK84. The genomic DNA of the ATCC13032 strain (=NBRC 12168 strain) was used as a template in amplification using primers ocJKT85 and ocJKT86 to obtain a DNA fragment containing the ORF region of GALT0 gene (SEQ ID NO: 1). The respective DNA fragments were purified from the obtained two types of PCR products using NucleoSpin Gel and PCR Clean-up (Takara Bio Inc.) and then ligated using In-Fusion HD Cloning Kit (Clontech Laboratories, Inc.) to prepare pHKBsacB-Δldh::GALT0. In this plasmid, ORF of the GALT0 gene was linked downstream of the plasmid region upstream of the ldh gene. The ECOS Competent E. Coli DH5a strain (Nippon Gene Co., Ltd.) was transformed with the obtained plasmid solution, and the cell solution was spread over LB agar medium containing kanamycin and left standing overnight at 37° C. The obtained colony was used as a template in colony PCR using Sapphire Amp (Takara Bio Inc.) as an enzyme. The primers used were ocJK105 and ocJK106, and the introduction of the DNA fragment of interest was confirmed. Transformants having the plasmid confirmed to harbor the gene were inoculated to 2 mL of LB liquid medium containing kanamycin and cultured overnight at 37° C. The plasmid was purified from this culture solution using NucleoSpin Plasmid EasyPure (Takara Bio Inc.) to obtain pHKBsacB-Δldh::GALT0.

2) Obtainment of Strain with GALT0 Gene Knocked in Cg3219 Locus

KC148 was transformed with the plasmid pHKBsacB-Δldh::GALT0 obtained as described above by electroporation (Bio-Rad Laboratories, Inc.). KC148Δldh::GALT0-sr was obtained by selection based on kanamycin resistance. As a result of performing PCR (Sapphire Amp) with the obtained colony as a template using primers ocJK107 and ocJK87, predictable results were obtained. The plasmid pHKBsacB-Δldh::GALT0 was therefore confirmed to be introduced into the cg3219 gene region by single-crossover homologous recombination. KC148Δldh::GALT0-sr was cultured in 1 mL of LB liquid medium for 24 hours, and a portion of the culture solution was smear-cultured on LB agar medium containing 20% sucrose to obtain a KC148Δldh::GALT0 strain. The GALT0 gene was confirmed to be knocked in the cg3219 locus, by colony PCR (Sapphire Amp) using primers ocJK107 and ocJK110. In addition, the kanamycin resistance gene and the sacB gene were confirmed to be also deleted.

(4) Preparation of Strain with GALT3 Gene Knocked in Cg3219 Locus

### 1) Preparation of Plasmid for Knocking GALT3 Gene in Cg3219 Locus

The enzyme for PCR used was PrimeSTAR Max DNA Polymerase (Takara Bio Inc.). A vector fragment was amplified with pHKBsacB-Δldh as a template using primers ocJK83 and ocJK84. A DNA fragment represented by SEQ ID NO: GALT3_nuc, which was prepared by artificial gene synthesis at Eurofins Genomics K.K., was used as a template in amplification using primers ocJKT87 and ocJKT88 to obtain a DNA fragment containing the ORF region of GALT3 gene (SEQ ID NO: 7). The respective DNA fragments were purified from the obtained two types of PCR products using NucleoSpin Gel and PCR Clean-up (Takara Bio Inc.) and then ligated using In-Fusion HD Cloning Kit (Clontech Laboratories, Inc.) to prepare pHKBsacB-Δldh::GALT3. In this plasmid, ORF of the GALT3 gene was linked downstream of the plasmid region upstream of the ldh gene. The ECOS Competent E. Coli DH5a strain (Nippon Gene Co., Ltd.) was transformed with the obtained plasmid solution, and the cell solution was spread over LB agar medium containing kanamycin and left standing overnight at 37° C. The obtained colony was used as a template in colony PCR using Sapphire Amp (Takara Bio Inc.) as an enzyme. The primers used were ocJK105 and ocJK106, and the introduction of the DNA fragment of interest was confirmed. Transformants having the plasmid confirmed to harbor the gene were inoculated to 2 mL of LB liquid medium containing kanamycin and cultured overnight at 37° C. The plasmid was purified from this culture solution using NucleoSpin Plasmid EasyPure (Takara Bio Inc.) to obtain pHKBsacB-Δldh::GALT3.

2) Obtainment of Strain with GALT3 Gene Knocked in Cg3219 Locus

KC148 was transformed with the plasmid pHKBsacB-Δldh::GALT3 obtained as described above by electroporation (Bio-Rad Laboratories, Inc.). KC148Δldh::GALT3-sr was obtained by selection based on kanamycin resistance. As a result of performing PCR (Sapphire Amp) with the obtained colony as a template using primers ocJK107 and ocJK98, predictable results were obtained. The plasmid pHKBsacB-Δldh::GALT3 was therefore confirmed to be introduced into the cg3219 gene region by single-crossover homologous recombination. KC148Δldh::GALT3-sr was cultured in 1 mL of LB liquid medium for 24 hours, and a portion of the culture solution was smear-cultured on LB agar medium containing 20% sucrose to obtain a KC148Δldh::GALT3 strain. The GALT3 gene was confirmed to be knocked in the cg3219 locus, by colony PCR (Sapphire Amp) using primers ocJK107 and ocJK110. In addition, the kanamycin resistance gene and the sacB gene were confirmed to be also deleted.

TABLE 1

| | | SEQ |
|---|---|---|
| Primer name | Sequence | ID NO |
| OT20 | AAAACGACGGCCAGTCAAATACGGCGCAGCCATCC | 13 |
| OT21 | GGACCGAAACGATCTGTTTTATCCAGCCTGACAAA | 14 |
| OT23 | CCGGCCCACGTGGCCATCTGGCTAGGGCAAAATCA | 15 |
| OT24 | ATCCTCTAGAGTCGAGACCACAAGAATTCCAAAAC | 16 |
| OT26 | GGCCAGCTGGGCCATTTAAA | 17 |
| OT27 | TTAATTAATTCCTCCTTTTATATTATTCAT | 18 |
| OT30 | GGAGGAATTAATTAAATGAATCATGTACCAGTGGC | 19 |
| OT31 | AAATTTAAACCTCCTTATACTTCGAAGCGTGGTA | 20 |
| OT32 | GGAGGTTTAAATTTATGAATCATGTACCGGTCGC | 21 |
| OT33 | GTGCCTCGGGTCTAAGGCCACGTGGGCCGGCGCGC | 22 |
| OT34 | TCGACTCTAGAGGATCTACT | 23 |
| OT35 | ACTGGCCGTCGTTTTACAAC | 24 |
| OT36 | GCACAGCCGTAGAAATC | 25 |
| OT37 | CGAAATCTCAGTTGGAGG | 26 |
| pHKPsacB-F2 | CTCGACTCTAGAGGATCTACTAGTCATATGGA | 27 |
| pHKPsacB-R2 | CTGCCTGCAGGCATGCAAGCTTGGCACTGGCCGTC | 28 |
| 3219-up-F | CATGCCTGCAGGCAGTTTCATACGACCACGGGCTACCCGAACGC | 29 |
| 3219-up-R | CCAAAGATTTTCGATCCCACTTCCTGATTTCCCTAACCGGACAA | 30 |
| 3219-down-F | ATCGAAAATCTTTGGCGCCTAGTTGGCGACGCAAGTGTTTCATT | 31 |
| 3219-down-R | TCCTCTAGAGTCGAGAAAACCCTGGTCACGGTGAATGCTCGGCG | 32 |
| sacB-1 | ATAGTTTGCGACAGTGCCGTCAGCG | 33 |
| 3219-up1500 | TTCGACGTCTCCCCACACCTCAGCCAAC | 34 |
| 3219-coloP-F | TAAAACAGCCAGGTTAGCAGCCGTAACCCA | 35 |
| 3219-coloP-R | GAGAATTTCGGCGTGCTCGTCGAGAATCTC | 36 |
| OCJK83 | TTTCGATCCCACTTCCTGATTTCCCTAAC | 37 |
| OCJK84 | ATCTTTGGCGCCTAGTTGGCGACGCAAG | 38 |
| OCJKT85 | GAAGTGGGATCGAAAATGCAGAAAAAACAACAGCTGAG | 39 |
| OCJKT86 | CTAGGCGCCAAAGATCTACTTCATTTCTGGAATACCTTTTCGAC | 40 |
| OCJK105 | ATAACACTTGGTCTGACCAC | 41 |
| OCJK106 | CTCCTTAAGCTACAAACACTC | 42 |
| OCJK107 | TTCGACGTCTCCCCACACCTCAG | 43 |
| OCJK87 | CTAGGCGCCAAAGATCTACTTCATTTCTGGAATACCTTTTCG | 44 |
| OCJK110 | GTCTTGTCGATGCAGACTGC | 45 |
| OCJKT87 | GAAGTGGGATCGAAAATGCAAAAGAACCAGCAGCTGTCTAC | 46 |
| OCJKT88 | CTAGGCGCCAAAGATTTACCCCATTTCGGGCATGCCTTTC | 47 |
| OCJK98 | CTAGGCGCCAAAGATTTACCCCATTTCGGGCATGCCTTTC | 48 |

### (5) Evaluation of Aromatic Compound Productivity

The KC148Δldh strain, the KC148Δldh::GALT0 strain, and the KC148Δldh::GALT3 strain were each streaked in LB plate and cultured at 30° C. for 3 days. Bacterial cells grown on the plate were inoculated to Round-Bottom Spitz (Eiken Chemical Co., Ltd.) charged with 4 mL of LB medium, and shake-cultured (precultured) at 200 rpm at 30° C. for 24 hours. Sodium benzoate was added to the CGXII medium shown in Table 2 so as to attain final concentration of 1 mM. A culture vessel of Bio Jr. 8 (ABLE Corporation) was charged with 100 mL of the resulting medium. 1 mL of the preculture solution was inoculated thereto, shake-cultured for 18 hours under conditions of 32° C., 700 rpm, and a ventilation volume of 100 mL/min, and evaluated for the productivity of aromatic compounds. The culture solution was appropriately diluted with dilute sulfuric acid, and the bacterial cells were removed by centrifugation, followed by the collection of a supernatant. The concentrations of gallic acid (GAL) and protocatechuic acid (also referred to as PCA) in the supernatant were quantified. The results are shown in Table 3. The KC148Δldh::GALT0 strain had 3.1 times the gallic acid concentration and 4.8 times the protocatechuic acid concentration of the KC148Δldh strain, and was thus confirmed to have the effect of improving the productivity of aromatic compounds. Likewise, the KC148Δldh::GALT3 strain had 2.7 times the gallic acid concentration and 3.1 times the protocatechuic acid concentration of the KC148Δldh strain, and was thus confirmed to have the effect of improving the productivity of aromatic compounds.

A GALT1 strain and a GALT2 strain harboring GALT1 or GALT2 gene instead of the GALT0 gene are also superior in the effect of improving the productivity of protocatechuic acid and gallic acid as compared to the strain before mutation.

TABLE 2
CGXII medium

| | | |
|---|---|---|
| Glucose | 50 | g/L |
| (NH₄)₂SO₄ | 20 | g/L |
| KH₂PO₄ | 1 | g/L |
| K₂HPO₄ | 1 | g/L |
| MgSO₄ 7H₂O | 0.25 | g/L |
| CaCl₂ 2H₂O | 10 | mg/L |
| FeSO₄ 7H₂O | 10 | mg/L |
| MnSO₄ 5H₂O | 10 | mg/L |
| ZnSO₄ 7H₂O | 1 | mg/L |
| CuSO₄ 5H₂O | 0.2 | mg/L |
| NiCl₂ 6H₂O | 0.02 | mg/L |
| Biotin (pH 7) | 0.2 | mg/L |
| Tryptone | 50 | g/L |

TABLE 3

| | GAL (mM) | PCA (mM) |
|---|---|---|
| KC148Δldh | 0.48 | 0.14 |
| KC148Δldh::GALT0 | 1.50 | 0.70 |
| KC148Δldh:GALT3 | 1.27 | 0.45 |

### Reference Example 1 Quantification of Gallic Acid and Protocatechuic Acid

Insoluble matter was removed from the collected supernatant using Acroprep 96-well filter plates (0.2 μm GHP filters, Nihon Pall Ltd.), and the reaction solution was subjected to HPLC.

The HPLC apparatus used was Chromaster (Hitachi High-Tech Corp.). Gradient elution was performed under conditions involving a flow rate of 1.0 mL/min and a column temperature of 40° C. using L-column ODS (4.6 mm I.D.×150 mm, Chemicals Evaluation and Research Institute, Japan), eluent A composed of a 0.1% phosphoric acid solution of 0.1 M potassium dihydrogen phosphate, and eluent B composed of 70% methanol. A UV detector (detection wavelength: 210) was used in detection.

### Reference Example 2 Analysis of Transmembrane Helix Structure Using Transmembrane Region Prediction Program

The polypeptide sequence of interest (here, GALT0) is transmitted to http://www.cbs.dtu.dk/services/TMHMM/. As a result, a transmembrane helix (transmembrane region) moiety is predicted, and moieties preceding and following this moiety are obtained as intracellular (inside) and extracellular (outside) moieties (FIG. 2). Prediction results are thereby obtained which show that GALT0 is a 12-pass transmembrane polypeptide having 12 transmembrane regions. Likewise, transmembrane regions of GALT1 or later can be predicted.

1. A method for producing an aromatic compound or a salt thereof, comprising culturing a transformed cell with enhanced expression of a multi-pass transmembrane polypeptide represented by (A) or (B):
(A) a polypeptide consisting of the amino acid sequence represented by SEQ ID NO: 2, or
(B) a polypeptide consisting of an amino acid sequence having at least 76% identity to the amino acid sequence represented by SEQ ID NO: 2.

2. The method according to claim 1, wherein the polypeptide consisting of an amino acid sequence having at least 76% identity to the amino acid sequence represented by SEQ ID NO: 2 in (B) is a polypeptide represented by any of (B1) to (B3):
(B1) a polypeptide consisting of the amino acid sequence represented by SEQ ID NO: 4 or an amino acid sequence having 90% or higher identity to the amino acid sequence represented by SEQ ID NO: 4,
(B2) a polypeptide consisting of the amino acid sequence represented by SEQ ID NO: 6 or an amino acid sequence having 90% or higher identity to the amino acid sequence represented by SEQ ID NO: 6, or
(B3) a polypeptide consisting of the amino acid sequence represented by SEQ ID NO: 8 or an amino acid sequence having 90% or higher identity to the amino acid sequence represented by SEQ ID NO: 8.

3. The method according to claim 1, wherein a polynucleotide encoding the multi-pass transmembrane polypeptide represented by (A) or (B) is contained in an expressible state.

4. The method according to claim 1, wherein the culture culturing is performed in the presence of a saccharide.

5. The method according to claim 1, wherein a host of the transformed cell is a microbial cell with improved 3-dehydroshikimic acid-producing activity.

6. The method according to claim 5, wherein the microbial cell with improved 3-dehydroshikimic acid-producing activity is a cell subjected to any one or more of genetic manipulations (i), (ii), (iii), or (iv):
(i) enhancement of one or more genes selected from the group consisting of dehydroshikimate dehydratase gene, dehydroquinate dehydratase gene, quinate dehydrogenase gene, and shikimate dehydrogenase gene,
(ii) enhancement of one or more genes selected from a gene group involved in the shikimic acid synthesis pathway consisting of 2-dehydro-3-deoxyarabinoheptonate aldolase gene, 3-dehydroquinate synthase gene, and shikimate dehydrogenase gene,
(iii) enhancement of one or more genes selected from a gene group involved in the pentose phosphate pathway consisting of glucose-6-phosphate dehydrogenase gene, 6-phosphogluconolactonase gene, phosphogluconate dehydrogenase gene, ribose-5-phosphate isomerase gene, ribulose-5-phosphate-3-epimerase gene, transketolase gene, and transaldolase gene, or
(iv) enhancement of a gene encoding a polypeptide having 3,4-dihydroxybenzoate hydroxylase activity.

7. The method according to claim 5, wherein the microbial cell is a coryneform bacterium.

8. The method according to claim 7, wherein the coryneform bacterium is a bacterium of the genus Corynebacterium.

9. The method according to claim 8, wherein the bacterium of the genus Corynebacterium is Corynebacterium glutamicum, Corynebacterium efficiens, Corynebacterium ammoniagenes, Corynebacterium halotolerance, Corynebacterium alkanolyticum, Corynebacterium crenatum, Corynebacterium crudilactis, or Corynebacterium callunae.

10. The method according to claim 8, wherein the bacterium of the genus Corynebacterium is Corynebacterium glutamicum.

11. The method according to claim 1, wherein the aromatic compound or the salt thereof is an aromatic compound derived from 3-dehydroshikimic acid or a salt thereof.

12. The method according to claim 11, wherein the aromatic compound or the salt thereof is gallic acid, protocatechuic acid, catechol, L-DOPA, 2,4-pyridinedicarboxylic acid, 2,5-pyridinedicarboxylic acid, 4-hydroxybenzoic acid, 4-aminobenzoic acid, 4-amino-3-hydroxybenzoic acid, or a salt thereof.

13. The method according to claim 11, wherein the aromatic compound or the salt thereof is gallic acid, protocatechuic acid, L-DOPA, 4-hydroxybenzoic acid, 4-amino-3-hydroxybenzoic acid, or a salt thereof.

14. The method according to claim 11, wherein the aromatic compound or the salt thereof is gallic acid, protocatechuic acid, or a salt thereof.

15. A transformed cell with enhanced expression of a multi-pass transmembrane polypeptide represented by (A) or (B):
(A) a polypeptide consisting of the amino acid sequence represented by SEQ ID NO: 2, or
(B) a polypeptide consisting of an amino acid sequence having at least 76% identity to the amino acid sequence represented by SEQ ID NO: 2, wherein
a microbial cell with improved 3-dehydroshikimic acid-producing activity is used as a host.

16. The transformed cell according to claim 15, wherein the microbial cell with improved 3-dehydroshikimic acid-producing activity is a cell subjected to any one or more of genetic manipulations (i), (ii), (iii), and (iv):
(i) enhancement of one or more genes selected from the group consisting of dehydroshikimate dehydratase gene, dehydroquinate dehydratase gene, quinate dehydrogenase gene, and shikimate dehydrogenase gene,
(ii) enhancement of one or more genes selected from a gene group involved in the shikimic acid synthesis pathway consisting of 2-dehydro-3-deoxyarabinoheptonate aldolase gene, 3-dehydroquinate synthase gene, and shikimate dehydrogenase gene,
(iii) enhancement of one or more genes selected from a gene group involved in the pentose phosphate pathway consisting of glucose-6-phosphate dehydrogenase gene, 6-phosphogluconolactonase gene, phosphogluconate dehydrogenase gene, ribose-5-phosphate isomerase gene, ribulose-5-phosphate-3-epimerase gene, transketolase gene, and transaldolase gene, or
(iv) enhancement of a gene encoding a polypeptide having 3,4-dihydroxybenzoate hydroxylase activity.

17. The transformed cell according to claim 15, wherein the polypeptide consisting of an amino acid sequence having at least 76% identity to the amino acid sequence represented by SEQ ID NO: 2 in (B) is a polypeptide represented by any of (B1) to (B3):
(B1) a polypeptide consisting of the amino acid sequence represented by SEQ ID NO: 4 or an amino acid sequence having 90% or higher identity to the amino acid sequence represented by SEQ ID NO: 4,
(B2) a polypeptide consisting of the amino acid sequence represented by SEQ ID NO: 6 or an amino acid sequence having 90% or higher identity to the amino acid sequence represented by SEQ ID NO: 6, or
(B3) a polypeptide consisting of the amino acid sequence represented by SEQ ID NO: 8 or an amino acid sequence having 90% or higher identity to the amino acid sequence represented by SEQ ID NO: 8.

18. The transformed cell according to claim 15, wherein a polynucleotide encoding the multi-pass transmembrane polypeptide represented by (A) or (B) is contained in an expressible state.

19. The transformed cell according to claim 15, wherein the microbial cell is a coryneform bacterium.

20. The transformed cell according to claim 19, wherein the coryneform bacterium is a bacterium of the genus Corynebacterium.

21. The transformed cell according to claim 20, wherein the bacterium of the genus Corynebacterium is Corynebacterium glutamicum, Corynebacterium efficiens, Corynebacterium ammoniagenes, Corynebacterium halotolerance, Corynebacterium alkanolyticum, Corynebacterium crenatum, Corynebacterium crudilactis, or Corynebacterium callunae.

22. The transformed cell according to claim 20, wherein the bacterium of the genus Corynebacterium is Corynebacterium glutamicum.


SEQUENCE LISTING (48 sequences)

SEQ ID NO: 1 - DNA, 1197 bp, Corynebacterium glutamicum
  source 1..1197: mol_type = genomic DNA
atgcagaaaaaacaacagctgagcaccgccctgattatgggattggcattattgtcagccagctccgcgctagcgactgatatgtatttgccggcaatgcctggtattgcggaagatttggggacaactgcaccgatggtgcagttaactctttcttcctttatggctggaatggcgattggccaattgatcattggtcctttgtcggatcaattgggaaggaaaggcctgctcgttgcaggtgcggtggctgcgctggtcgctagtgtggtgtgcgcgctggcgccgtcgataagcgtattagtgatcgcacgcctggtgcaggggcttggcggcggtgcgtgcgtggtgattgcgcgcgcgatcgtgccagaccttgaacgcggacaaaaggctgcgcacgcctttgcactgctgatgattattcagggaattgctcccgtggtagctccgctcattggtggtgtgctggtcgggccttttggctggcggggaattttctgggcacttgcactggtgaattttgcgcagctgcttgttgctttgctgcagattaaggagtcgaagccagttgaagagcgtaccgcagcaggacttggcggaatgctgtccaactatgtctttgtgctgaagaatcctcaatttttggcatatgtattcacattggggctgtcttttggggcgatgttctcctacatttcggcgtcgccgttcgtgctgcagaatcaaatgggcattccggtactgctgtattccattattttcggagtgaatgcttttggtttgattgtgggcggaatggtcaataggcgacttctgcagcggattcatccacaccgcatcatgcaaactgtgctggccagttttactgtgctgtgtgcgcttttgctgattgaagtgctgtttattaattggataccgctgttcctgttgctgctgtttcttatcgtttcccatattccgatggttatggctaacgcgacagctctgggaactgaagtggtgcgaagcagggcgggatcgggttctgcaattttgggtttcgtgcaattcacgatgggtgctttggtgagttcactggtcggattaggctctgataaggctttgactatgggaatcgcaatgactgcttgtgcactgctggcgtgtgggtgtgcgtacctggcaggtcgaaaaggtattccagaaatgaagtag

SEQ ID NO: 2 - protein, 398 aa, Corynebacterium glutamicum
MQKKQQLSTALIMGLALLSASSALATDMYLPAMPGIAEDLGTTAPMVQLTLSSFMAGMAIGQLIIGPLSDQLGRKGLLVAGAVAALVASVVCALAPSISVLVIARLVQGLGGGACVVIARAIVPDLERGQKAAHAFALLMIIQGIAPVVAPLIGGVLVGPFGWRGIFWALALVNFAQLLVALLQIKESKPVEERTAAGLGGMLSNYVFVLKNPQFLAYVFTLGLSFGAMFSYISASPFVLQNQMGIPVLLYSIIFGVNAFGLIVGGMVNRRLLQRIHPHRIMQTVLASFTVLCALLLIEVLFINWIPLFLLLLFLIVSHIPMVMANATALGTEVVRSRAGSGSAILGFVQFTMGALVSSLVGLGSDKALTMGIAMTACALLACGCAYLAGRKGIPEMK

SEQ ID NO: 3 - DNA, 1197 bp, synthetic construct
  source 1..1197: mol_type = other DNA
atgcagaagaagcagcagctctccaccgcactcattatgggattggctctcttgtccacctcttcagcgcttgccaccgacatgtatctgcctgctatgccagggattgctgaggacttgggtacgaccgcgccaatggttcagttgaccttgtccagcttcatggctggcatggcaatcggacagctcatcatcggtcctctgagcgatcaggttggccgcaaaggccttctggtcgcaggagcagtggcagccctggttgcctctgtcgtgtgtgcgcttgctccttcgattagcgttttggtgatcgcacgtctggtccaagggctgggtggtggcgcttgcgtggttatcgcacgcgctattgtacccgatcttgaacgcggccagaaagccgctcatgcgttcgccttgctgatgatcattcaaggcattgcaccggtggttgcgccgctgattggtggcgtcctcgtcggcccttttggttggcgtggcatcttctgggctctggcactggtgaactttgcgcagctgttggtcgcactccttcagattaaggagtccaaaccagtcgaagaacgtacagctgcaggactcggcggtatgctctccaactacgttttcgtgctcaagaatccgcagtttcttgcttacgtgttcaccctgggtctgtcgtttggcgcgatgttctcctacatctccgcttctcccttcgttctccagaaccagatgggtatcccagtcctgctgtactccatcatctttggggtgaatgccttcgggcttatcgtaggtggcatgatcaaccggcgcttgctgcaacgtatccacccacaccgcatcatgcaaaccgttctcgcctcattcacggttctttgcgctctgctcctgatcgaagtgctgttcatcaactggatcccactgttcctgctgttgctcttccttatcgtatcgcacattccgatggtgatggccaacgctactgcgctgggcactgaggttgtacggagccgagcaggatctggatctgcaattctgggtttcgtccaattctccatgggtgccctggtgtcctcactcgtcggtcttggctccgataaggcggtcaccatgggctttgccatgactggctgcgcattgctggcgtgtggctgcgcctatcttgcaggacgcaagggaatccccgagatgcgctaa

SEQ ID NO: 4 - protein, 398 aa, Corynebacterium crenatum
MQKKQQLSTALIMGLALLSTSSALATDMYLPAMPGIAEDLGTTAPMVQLTLSSFMAGMAIGQLIIGPLSDQVGRKGLLVAGAVAALVASVVCALAPSISVLVIARLVQGLGGGACVVIARAIVPDLERGQKAAHAFALLMIIQGIAPVVAPLIGGVLVGPFGWRGIFWALALVNFAQLLVALLQIKESKPVEERTAAGLGGMLSNYVFVLKNPQFLAYVFTLGLSFGAMFSYISASPFVLQNQMGIPVLLYSIIFGVNAFGLIVGGMINRRLLQRIHPHRIMQTVLASFTVLCALLLIEVLFINWIPLFLLLLFLIVSHIPMVMANATALGTEVVRSRAGSGSAILGFVQFSMGALVSSLVGLGSDKAVTMGFAMTGCALLACGCAYLAGRKGIPEMR

SEQ ID NO: 5 - DNA, 1197 bp, synthetic construct
  source 1..1197: mol_type = other DNA
atgcagaagaagcagcagttgaccaccgccctgattatggggctcgctttgctgtctgcatcctctgccttgtccaccgacatgtaccttccagcgatgccaggcatcgcagaagatctgaacacgactgcaccaatggtgcagctgaccctgtcctcctacatggctggtatggccgttgggcaacttatcatcgggcctctttccgaccaacttgggcgcaagtccctgctcgtggcaggagcggtcgctgcgctggttgcatcctttgtgtgcgccctcgcaccctccatctcggtgctcatcatcgcacgccttgtgcaaggcctgggaggtggagcatgcgttgtgattgcacgggctatcgtcccagatctcgaacgcggtcagaaagcagcgcaagcattcgccctgctcatgatcatccaaggagtagcaccggttgttgccccgcttattggcggaatgctggtcggtccctttggttggcgtggaatcttttgggcgctggctcttatcaaccttgcgcagttgctcgttgcttctttccagatcaaggagagcaaaccggtagaggaacgcactgccgcaggcttgatgggcatgctctcgaactacaccttcgtcctgaagaaccctcagttcttggcctatgtgttcacactgggtctgtcgtttggtgcgatgttctcctacatctcagcttctccattcgtgctgcagaaccagatgggcatccccgtcctgctgtactcagtcatcttcggtgtcaatgcttgtggcctcatcattggcggcatgattaaccgccgactgttgcgttccgtgcatcctcaccgcattatgcagttcgtgctggcttcctttaccgtcctctgcgctgtgctcctcatcgaggttctggtaatccactggactccactgttcctcgtcttgctgttcctgcttgtgtctcacattccgatggtcatggccaatgctacggctcttggtaccgaagttgtccgctctcgggctggtagcggatcagccattcttggtttcgttcagttcgccatgggcgcactggtttcaagcctcgttggcttgggctcggataaggcagtctccatggcgtttagcatgaccgtatgcgccattctggcgtgtggctgcgcttatttggcgggccgtaaaggcgtgcctgagatgaagtaa

SEQ ID NO: 6 - protein, 398 aa, Corynebacterium glutamicum
MQKKQQLTTALIMGLALLSASSALSTDMYLPAMPGIAEDLNTTAPMVQLTLSSYMAGMAVGQLIIGPLSDQLGRKSLLVAGAVAALVASFVCALAPSISVLIIARLVQGLGGGACVVIARAIVPDLERGQKAAQAFALLMIIQGVAPVVAPLIGGMLVGPFGWRGIFWALALINLAQLLVASFQIKESKPVEERTAAGLMGMLSNYTFVLKNPQFLAYVFTLGLSFGAMFSYISASPFVLQNQMGIPVLLYSVIFGVNACGLIIGGMINRRLLRSVHPHRIMQFVLASFTVLCAVLLIEVLVIHWTPLFLVLLFLLVSHIPMVMANATALGTEVVRSRAGSGSAILGFVQFAMGALVSSLVGLGSDKAVSMAFSMTVCAILACGCAYLAGRKGVPEMK

SEQ ID NO: 7 - DNA, 1197 bp, synthetic construct
  source 1..1197: mol_type = other DNA
atgcaaaagaaccagcagctgtctaccacgctcatcatgggacttgcgttgctttccgcgtcatcagccctggcaaccgacatgtaccttcctgccttgcctgggattagccaggatcttggcaccactgcggcatccgttcaactgaccctttcctcgttcatggcaggcatggcagtcggtcagctcatcatcggcccactctcggatcagttggggcgcaagggtttcctggttgctggtgcggtggcagccttgttggcatcggttgtttgcgcaatggccccttctatcagcatcctcgttctcgctcgcttggtccagggccttggtggcggagcctgcgtagtgattgctcgtgctattgtgccggatctggcgcgaggtcaagttgccgctcaagcattcgcactgctcatgatcatccagggagtagctccagtagtggcacccctgattggcggtgttttggtcggtccgttcggatggcgtggcatcttctgggctctcgcgttggtgaacctcgctcagctgcttgtcgcacttctgctggtcaaggagtccaagccagtggaagaacgcacagtggctgggcttaagggcatgctgtccaactatcgcttcgttttgtccaatccccaattcctcgcctacgtctttaccctgtccttggctttcggcgcaatgttctcctacatttccgcctctcctttcgtgctccagaaccagatgggcatttccgttctggcatattccgtcatctttggcgtgaacgcgtgtggtcttatcatcggaggtatgatcaaccgtcggcttctgcttcgcctccatccacaccgcatcatgcagactgtgctcatcagcttcaccgtactctgcgtcatcctgctggtggaggttctgttcatccagtggtcaccactgtttctggcactgctctttctgattgtctcccacattccgatgattatggccaatgccacggcgttgggtgtgggcgttgtccgctctcgtgctggctcgggatctgcaatcctgggatttatgcagttctctatgggcgctctggtcagcccactgatgggtctgggtgccgacaaagccgtgactatggcggtttcaatggtctgctgcgctctgctcgcttccggctgtgcgtttctggctggacggaaaggcatgcccgaaatggggtaa

SEQ ID NO: 8 - protein, 398 aa, Corynebacterium crudilactis
MQKNQQLSTTLIMGLALLSASSALATDMYLPALPGISQDLGTTAASVQLTLSSFMAGMAVGQLIIGPLSDQLGRKGFLVAGAVAALLASVVCAMAPSISILVLARLVQGLGGGACVVIARAIVPDLARGQVAAQAFALLMIIQGVAPVVAPLIGGVLVGPFGWRGIFWALALVNLAQLLVALLLVKESKPVEERTVAGLKGMLSNYRFVLSNPQFLAYVFTLSLAFGAMFSYISASPFVLQNQMGISVLAYSVIFGVNACGLIIGGMINRRLLLRLHPHRIMQTVLISFTVLCVILLVEVLFIQWSPLFLALLFLIVSHIPMIMANATALGVGVVRSRAGSGSAILGFMQFSMGALVSPLMGLGADKAVTMAVSMVCCALLASGCAFLAGRKGMPEMG

SEQ ID NO: 9 - DNA, 1155 bp, synthetic construct
  source 1..1155: mol_type = other DNA
atggctcttctggcagcctctccagcattgtctacggacatgtacttgcctgcgatgccagggattgccgcggatctgaacaccacaaccccactggttcagctgacactctccgccttcatggccggtatggcaatcggacagctgatcattggccccatttccgaccaactgggtcgcaaaggcttgcttatcggtggtgcagtcgcagccctgattgcatccgtgctgtgtgctgttgctccctcgattagcgtcctcatcgcagcacgcatcctccaaggacttggcggcggtgcgtgcgttgtcatctcccgtgccattgtaccggatctggcacgcgggaagaaggcagcgcaagccttcgccctcctcatgatcatccaaggcgtcgctcctgtggctgcgccgttgttcggcggagttctcgtggaacacgttggctggcgcggtatcttctggaccttggctctgatcaacgtggcgcagttgatggtcgtcatcttcggcatccgcgagtcgaaacccgtcgaagaacgctctgctgctggtgtgcgtggtatgctggcgaactacgcatttgtgcttggcaatcgacagttcctggcctatgtcttgaccctggctttcgcttttggcgcgcttttctcctacatttcggcctctccattcgttatccagacccagatgggcatgtccgttggagtgtttagcctgatcttcggagttaacgcttgtggcctgattatcggcgggatggtgaaccgtcggctgttgcagaaggttcatgcccacaaggttatgcggaccgtactcctgagctttgtgatcctgtgcctcctgctccttctcgaggtgctcttcgtacagtggactcctctctttctggtccttttgttcctgcttatgtcacacatcccgatgattatggcgaatgccactgcgctgggcactgaggtagtgcgtgcacgagctggttccggatcagcaatccttggcttcacgcagttttccatggcagctcttgtctccccactcgtcggtttggggtcagataaggcacttaccatggctctggcgatggctatctccgcactgtgcgctttgggtttcagctatctcgcaggacgcaaaggtctgcctaccatggcctaa

SEQ ID NO: 10 - protein, 384 aa, Corynebacterium callunae
MALLAASPALSTDMYLPAMPGIAADLNTTTPLVQLTLSAFMAGMAIGQLIIGPISDQLGRKGLLIGGAVAALIASVLCAVAPSISVLIAARILQGLGGGACVVISRAIVPDLARGKKAAQAFALLMIIQGVAPVAAPLFGGVLVEHVGWRGIFWTLALINVAQLMVVIFGIRESKPVEERSAAGVRGMLANYAFVLGNRQFLAYVLTLAFAFGALFSYISASPFVIQTQMGMSVGVFSLIFGVNACGLIIGGMVNRRLLQKVHAHKVMRTVLLSFVILCLLLLLEVLFVQWTPLFLVLLFLLMSHIPMIMANATALGTEVVRARAGSGSAILGFTQFSMAALVSPLVGLGSDKALTMALAMAISALCALGFSYLAGRKGLPTMA

SEQ ID NO: 11 - DNA, 1188 bp, synthetic construct
  source 1..1188: mol_type = other DNA
atgaatcatgtaccagtggcaattattggcgcaggaccagcaggactaaccctcgcccacctcctccaccttcaaggtgtggaatcaatcgtctttgaatcccgcgcccgcaaggacgtcgaagaaaccgtccgagcaggcatcctggaacaaggcaccctgaatctgatgcgcgaaaccggagtcggcgcacgcatggaagcagaagccgatcacgatgaagcaatcgacatctccatcaacaatgagcgcacccgcattccgctgaccgaactcaccggccacaaggttgcgatctacccgcagcacgaatacctcaaagatttcattgccaagcgcatcgaagatggcggcgaactccttttcaccaccactgttgattccgtagaaaactacgaaggcgacctcgccaaggtgacctacaccgaagccgatggttcctccaccaccatcaccgccgactacgtcatcgcagctgacggctccaactccccttaccgcaagctgatcaccgaagacggtggcgtgcgcgcccgccatgaatacccttacgcatggttcggcattttggtggaagcaccaaaaacccaaaaggaagtcatctacgcaacccaccctgagggctttgcgctgatctccacccgtaccgatgaaatccagcgctactacctgcagtgcaaccctgacgacaccccagacatgtggcccgatgaccgcatttgggaacagctgcacctgcgtgcggactcccctggcatcaccgtgtctgaagggcgcatctttgacaaggccgtgctgcgtttccgctccgcggtcaccgaaccaatgcaaaagggacgcctcttccttgctggcgatgctgcacacaccgtgccgccaaccggagctaagggcctcaacttggctgttgccgatgtctcagtactcgcgccagcactggttcgtgccctgaagaagaaggacaccggcttgctcgatagctacacctccctggcagtcccccgcatctggaaagcacagcacttctcctactggatgagctccatgctccacgcagtacccggcgaagatcactttgccacccagcgccgattcgctgaattgcgctccgtcctagaatcccaatccggccaacgctacctcgcagagcagttcgttgggcgcgacctaccacgcttcgaagtataa

SEQ ID NO: 12 - DNA, 1280 bp, synthetic construct
  source 1..1280: mol_type = other DNA
atgaatcatgtaccggtcgctatcatcggcgctggtccggctggtctgacgctggctcacctgctgcacctgcaaggcgttgaaagcatcgtttttgaatctcgtacgcgcaaagatgttgaagaaaccgtccgtgcgggcattctggaacagggtacgctgaacctgatgcgtgaaaccggcgtcggtgcacgcatggaagcggaagccgatcatgacgaagctattgatatcagcattaacaatgaacgtacccgtatcccgctgacggaactgaccggtcataaagtggcaatttatccgcaacacgaatacctgaaagattttatcgctaaacgtattgaagacggcggtgaactgctgttcaccacgaccgtcgatagtgtggaaaactatgaaggcgacctggcgaaagtgacgtacaccgaagccgatggtagctctacgaccatcacggcggattatgttattgcggccgacggttcaaattcgccgtaccgcaaactgatcaccgaagatggcggtgttcgtgcacgccatgaatatccgtacgcttggtttggcatcctggtcgaagccccgaaaacgcagaaagaagtgatttatgcgacccacccggaaggtttcgccctgatcagcacgcgtaccgatgaaattcagcgctattacctgcaatgcaacccggatgacaccccggacatgtggccggatgaccgtatttgggaacagctgcatctgcgcgcggattcaccgggcatcaccgtgtcggaaggtcgtatttttgacaaagcagtgctgcgtttccgcagtgctgttaccgaaccgatgcaaaaaggccgcctgtttctggcgggtgatgcagctcacacggttccgccgaccggtgcaaaaggtctgaatctggcagttgctgacgtgtccgttctggcaccggcactggtccgtgcactgaagaaaaaagatacgggcctgctggacagttataccagcctggcggtgccgcgcatttggaaagcccagcacttcagctactggatgagttccatgctgcatgcagtcccgggtgaagatcactttgcaacccagcgtcgcttcgctgaactgcgctctgtgctggaaagccaatctggtcaacgctatctggctgaacaattcgtcggtcgtgatctgccgcgctttgaagtgtaagaggtttaaacaagcggccgcgatatcgttgtaaaaaaccccgctccggcggggttttttgtatctggccaggcgcgccggcccacgtggcc

SEQ ID NO: 13 - DNA, 35 bp, synthetic construct
  source 1..35: mol_type = other DNA
aaaacgacggccagtcaaatacggcgcagccatcc

SEQ ID NO: 14 - DNA, 35 bp, synthetic construct
  source 1..35: mol_type = other DNA
ggaccgaaacgatctgttttatccagcctgacaaa

SEQ ID NO: 15 - DNA, 35 bp, synthetic construct
  source 1..35: mol_type = other DNA
ccggcccacgtggccatctggctagggcaaaatca

SEQ ID NO: 16 - DNA, 35 bp, synthetic construct
  source 1..35: mol_type = other DNA
atcctctagagtcgagaccacaagaattccaaaac

SEQ ID NO: 17 - DNA, 20 bp, synthetic construct
  source 1..20: mol_type = other DNA
ggccagctgggccatttaaa

SEQ ID NO: 18 - DNA, 30 bp, synthetic construct
  source 1..30: mol_type = other DNA
ttaattaattcctccttttatattattcat

SEQ ID NO: 19 - DNA, 35 bp, synthetic construct
  source 1..35: mol_type = other DNA
ggaggaattaattaaatgaatcatgtaccagtggc

SEQ ID NO: 20 - DNA, 34 bp, synthetic construct
  source 1..34: mol_type = other DNA
aaatttaaacctccttatacttcgaagcgtggta

SEQ ID NO: 21 - DNA, 34 bp, synthetic construct
  source 1..34: mol_type = other DNA
ggaggtttaaatttatgaatcatgtaccggtcgc

SEQ ID NO: 22 - DNA, 35 bp, synthetic construct
  source 1..35: mol_type = other DNA
gtgcctcgggtctaaggccacgtgggccggcgcgc

SEQ ID NO: 23 - DNA, 20 bp, synthetic construct
  source 1..20: mol_type = other DNA
tcgactctagaggatctact

SEQ ID NO: 24 - DNA, 20 bp, synthetic construct
  source 1..20: mol_type = other DNA
actggccgtcgttttacaac

SEQ ID NO: 25 - DNA, 17 bp, synthetic construct
  source 1..17: mol_type = other DNA
gcacagccgtagaaatc

SEQ ID NO: 26 - DNA, 18 bp, synthetic construct
  source 1..18: mol_type = other DNA
cgaaatctcagttggagg

SEQ ID NO: 27 - DNA, 32 bp, synthetic construct
  source 1..32: mol_type = other DNA
ctcgactctagaggatctactagtcatatgga

SEQ ID NO: 28 - DNA, 35 bp, synthetic construct
  source 1..35: mol_type = other DNA
ctgcctgcaggcatgcaagcttggcactggccgtc

SEQ ID NO: 29 - DNA, 44 bp, synthetic construct
  source 1..44: mol_type = other DNA
catgcctgcaggcagtttcatacgaccacgggctacccgaacgc

SEQ ID NO: 30 - DNA, 44 bp, synthetic construct
  source 1..44: mol_type = other DNA
ccaaagattttcgatcccacttcctgatttccctaaccggacaa

SEQ ID NO: 31 - DNA, 44 bp, synthetic construct
  source 1..44: mol_type = other DNA
atcgaaaatctttggcgcctagttggcgacgcaagtgtttcatt

SEQ ID NO: 32 - DNA, 44 bp, synthetic construct
  source 1..44: mol_type = other DNA
tcctctagagtcgagaaaaccctggtcacggtgaatgctcggcg

SEQ ID NO: 33 - DNA, 25 bp, synthetic construct
  source 1..25: mol_type = other DNA
atagtttgcgacagtgccgtcagcg

SEQ ID NO: 34 - DNA, 28 bp, synthetic construct
  source 1..28: mol_type = other DNA
ttcgacgtctccccacacctcagccaac

SEQ ID NO: 35 - DNA, 30 bp, synthetic construct
  source 1..30: mol_type = other DNA
taaaacagccaggttagcagccgtaaccca

SEQ ID NO: 36 - DNA, 30 bp, synthetic construct
  source 1..30: mol_type = other DNA
gagaatttcggcgtgctcgtcgagaatctc

SEQ ID NO: 37 - DNA, 29 bp, synthetic construct
  source 1..29: mol_type = other DNA
tttcgatcccacttcctgatttccctaac

SEQ ID NO: 38 - DNA, 28 bp, synthetic construct
  source 1..28: mol_type = other DNA
atctttggcgcctagttggcgacgcaag

SEQ ID NO: 39 - DNA, 38 bp, synthetic construct
  source 1..38: mol_type = other DNA
gaagtgggatcgaaaatgcagaaaaaacaacagctgag

SEQ ID NO: 40 - DNA, 44 bp, synthetic construct
  source 1..44: mol_type = other DNA
ctaggcgccaaagatctacttcatttctggaataccttttcgac

SEQ ID NO: 41 - DNA, 20 bp, synthetic construct
  source 1..20: mol_type = other DNA
ataacacttggtctgaccac

SEQ ID NO: 42 - DNA, 21 bp, synthetic construct
  source 1..21: mol_type = other DNA
ctccttaagctacaaacactc

SEQ ID NO: 43 - DNA, 23 bp, synthetic construct
  source 1..23: mol_type = other DNA
ttcgacgtctccccacacctcag

SEQ ID NO: 44 - DNA, 42 bp, synthetic construct
  source 1..42: mol_type = other DNA
ctaggcgccaaagatctacttcatttctggaataccttttcg

SEQ ID NO: 45 - DNA, 20 bp, synthetic construct
  source 1..20: mol_type = other DNA
gtcttgtcgatgcagactgc

SEQ ID NO: 46 - DNA, 41 bp, synthetic construct
  source 1..41: mol_type = other DNA
gaagtgggatcgaaaatgcaaaagaaccagcagctgtctac

SEQ ID NO: 47 - DNA, 40 bp, synthetic construct
  source 1..40: mol_type = other DNA
ctaggcgccaaagatttaccccatttcgggcatgcctttc

SEQ ID NO: 48 - DNA, 40 bp, synthetic construct; identical to SEQ ID NO: 47
  source 1..40: mol_type = other DNA